(19) United States
(12) Patent Application Publication
(10) Pub. No.: US 2007/0141055 A1
(43) Pub. Date: Jun. 21, 2007

(54) Methods and compositions for protein-hydroxy apatite complexes and their application in testing and modulating immunological system including a novel in vitro test for the detection of antibodies against calcium binding protein-hydroxy apatite complexes

(76) Inventors: E. Olavi Kajander, Lakeland, FL (US); Neva Ciftcioglu, League City, TX (US); Katja M. Aho, Siilinjarvi (FI)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 11/491,519

(22) Filed: Jul. 24, 2006

Related U.S. Application Data
(63) Continuation of application No. 11/268,727, filed on Nov. 8, 2005, now abandoned.
(60) Provisional application No. 60/625,572, filed on Nov. 8, 2004.

(51) Int. Cl. A61K 39/395; A61K 33/42; C07K 16/18

(52) U.S. Cl. 424/145.1; 514/12; 424/602; 530/388.25

(57) ABSTRACT

The invention relates to methods and compositions for the manufacture and use of novel hydroxy apatite (HA)-calcium binding protein (CaBP) complexes (CaBP-HA complex) that are useful as antigen preparations for immunological assays for the diagnosis, prognosis, and monitoring of mammalian diseases. The CaBP-HA complexes may be manufactured using synthetic HA that is subjected to serum proteins or by harvesting nanobacteria (NB), also called calcifying nano-particles (CNP), from a mammal. The hydroxy apatite is prepared by incubation with appropriate proteins resulting in conformational changes in said bound proteins. Secondary conformational changes occur when said CaBP-HA complex is subjected to enzymes such as transglutaminase thereby creating covalently bound neoepitopes. The CaBP-HA complex may also contain lipopolysaccharide binding protein (LPSBP) providing for anti-CaBP-LPSBP-HA antibodies detection indicative of disease. Detection of anti-CaBP-HA antibodies in a mammal is via enzyme linked immunosorbent assay.

### CROSS-REFERENCES

This application claims the benefit of the filing date of U.S. Provisional Patent Application No 60/625,572, filed Nov. 8, 2004.

### FIELD OF INVENTION

The invention relates generally to a method and kit providing for the rapid in vitro detection of antibodies directed against proteins that have undergone calcium mediated conformational changes in mammals. More particularly, the invention discloses a method for the detection of host antibodies against proteins that have conformational changes as a result of binding to hydroxy apatite or mineral calcium substrates. Calcium phosphate mineral surface offers very high binding capacity and very high binding affinity for proteins interacting with calcium. Because calcium phosphate mineral (apatite) is transparent to light, this substrate is applicable with optical quantification devices as described in the body of the invention. These protein hydroxy apatite preparations may be immobilized onto a solid support which can be used in a test kit on a routine basis in a laboratory setting for diagnostic and prognostic purposes and methods utilizing the kits to rapidly detect immune response in a mammal.

### BACKGROUND OF INVENTION

The formation of discrete and organized inorganic crystalline structures within macromolecular extra cellular matrices is a widespread biological phenomenon generally referred to as biomineralization. One example of biomineralization is the formation of calcium phosphate. When calcium phosphate is deposited in tissue, it is known as calcification. Mammalian bone and dental enamel are examples of calcification. Greater than 50% of all known diseases that affect mammals, and in particular humans, are caused by or associated with the deposition of mineral calcium. Mineral calcium, comprised of calcium phosphate, also called hydroxy apatite, is not the healthy process that builds bones and teeth, but instead it is found in disease. Pathological calcification is found in a variety of diseases. While the cause of pathological calcification remains unknown, researchers have discovered a common link in each of these diseases—that is, the presence of very small mineral-associated bacteria-like particles called Nanobacteria or Calcifying Nano-particles (NB/CNP).

Pathological calcification resulting in the deposition of hydroxy apatite surfaces within a mammal creates a unique substrate for the collection of calcium binding proteins (CaBP). It is well known to those in the art that CaBPs will bind to calcium phosphate hydroxy apatite. There are greater than 3 thousand CaBPs and fragments thereof presently listed on the Swiss-Prot Protein knowledgebase. Hydroxy apatite exposed to blood will bind calcium binding proteins such as prothrombin, C-reactive protein, and Matrix GLA Protein. Clinical experience in cardiovascular medicine suggests that contact of blood with an exposed hydroxy apatite surface leads to thrombi. Recent coronary calcification scoring data support that experience, because positive scores are good biomarkers to predict future atherosclerotic thrombotic events, such as myocardial infarcts and strokes. Many studies have also shown that patients with calcification-associated diseases such as atherosclerosis, kidney and autoimmune diseases, diabetes and cancer often have abnormal ongoing blood coagulation and thrombosis.

Calcium phosphate can mediate conformational change in many cases in three-dimensional conformation of the proteins due to its multiple interactions with the proteins. Such conformational forms are found normally in bone and temporarily inside cells and function in various biological regulatory systems as rapid switches controlling metabolic or physiological pathways and reactions. Calcium phosphate mineral is not a normal constituent in blood or soft tissue, and in bone is protected by endosteum from direct exposure to blood and immunological system. When calcium phosphate particles are exposed to blood, the immunological system can “see” the new protein conformations stabilized on the surface of the particles, and react against them. This will create autoantibody formation and autoantibodies are well known to mediate pathological processes in autoimmune diseases as well as in other diseases where novel protein conformations are exposed and become immunologically recognized. Autoimmunity has been recognized also in atherosclerosis.

Four clotting factors (Factor II, Factor VII, Factor IX, Factor X) are calcium-binding proteins with a Gla domain and other calcium binding sites. For example, Gla-domains bind avidly to hydroxy apatite/calcium phosphate similarly to another Gla-protein family, consisting of matrix Gla-protein, osteopontin, osteocalcin and osteonectin, known to regulate calcification as part of the calcification-defense system [(R. W. Romberg, P. G. Werness, B. L. Riggs, K. G. Mann, Biochemistry 25, 1176 (1986).) & G. E. Donley, L. A. Fitzpatrick, Trends Cardiovasc. Med. 8, 199 (1998).]. Fibrin, fibrinogen, factor XIIIa, fragments of factor II, thrombin, prothrombin Fragment I all are examples of CaBPs.

Hydroxy apatite surfaces serve a dual function as a suitable substratum and activator in mammalian blood or serum for CaBPs. The significance of this for human (patho)physiology is high because there are many situations where hydroxy apatite can have contact with blood. Namely, 1) acutely during bone fracture, bone surgery and dental surgery 2) artificially with the introduction of uncoated implants, fillers and apatite adjuvants, 3) chronically with the growth of calcium phosphate deposits in atherosclerotic vessels, catastrophically with rupture of vulnerable plaque, and via cell death, exposing pathological calcification (e.g., Randall's plaque, atherosclerotic plaque and cancer calcifications) and stones, 4) hematologically with calcium phosphate/hydroxy apatite macromolecular complexes in blood of animals suffering massive bone degradation and 5) systemically with NB/CNPs.

CaBP binding to hydroxy apatite is mediated by the presence of free calcium in the blood or serum. Free calcium differs from mineral calcium hydroxy apatite in the body. Free calcium atoms have a net 2 positive charge at the physiological pH. Free calcium is extremely well kept in the mammalian body with typical concentrations in human blood of approximately 1 mM. Elevated levels of free calcium are indicative of abnormal activity or disease. In the presence of free calcium, CaBPs will bind to calcium phosphate mineral, if available, and thereby experience a conformational change. Therefore, the detection of antibodies present in the body of the mammal against the conformational changed CaBPs may be useful in the diagnosis and prognosis of disease or pathology involving pathologic calcification.

The conformation change in CaBPs upon binding to calcium is well documented and known to those in the art. As stated, calcium ions directly affect the binding characteristics of CaBPs. By calcium binding of CaBPs with various affinities, calcium ions mediate calcium-dependent functions by inducing conformational changes, stabilizing their target proteins, protecting CaBPs from proteolytic degradation, and regulating domain interactions. For example, calmodulin, an intracellular EF-hand calcium binding protein, regulates more than 100 proteins upon calcium-dependent conformational change. Cadherin, an extracellular non-EF-hand calcium binding protein with a structural topology similar to that of CD2, plays essential roles in controlling the development and maintenance of tissues.. (Jenny J Yang Laboratory)

M. R. Nelson and W. J. Chazin of the Dep. of Molecular Biology, The Scripps Research Institute published a detailed analysis of the calcium binding-induced conformational changes in the representative calcium sensors calmodulin (CaM) and troponin entitled An interaction-based analysis of calcium-induced conformational changes in Ca(2+) sensor proteins wherein they claim that the calcium-induced conformational changes in these proteins are dominated by reorganization of the packing of the four helices within each domain and that comparison of the closed and open conformations confirms that calcium binding causes openings within each of the EF-hands. Aalim M. Weljie et al., Protein conformational changes studied by diffusion NMR spectroscopy: Application to helix-loop-helix calcium binding proteins utilized pulsed-field gradient (PFG) diffusion NMR spectroscopy studies conducted with several helix-loop-helix regulatory Ca²⁺-binding proteins to characterize the conformational changes associated with Ca²⁺-saturation and/or binding targets.

### BRIEF SUMMARY OF THE INVENTION

Disclosed are methods and compositions for detecting and assessing the significance of antibodies to calcium binding proteins (CaBPs) bound to hydroxy apatite substrates thereby forming a complex wherein said calcium binding protein has undergone a conformation change. The disclosed methods and compositions generally involve the manufacture of a hydroxy apatite substrate containing bound and conformationally changed calcium binding proteins (antigens) or CaBP-HA complexes (CaBP-HA complex hereafter).

The disclosed methods and compositions of the present invention describe techniques that can be used to measure primary as well as secondary changes as they occur during the binding of protein by a hydroxy apatite substrate. Primary changes occur upon initial interaction of a CaBP or fragment thereof, with calcium phosphate, that are, for example, physiological changes. Secondary changes are cross-linking type of changes that are caused by other proteins or enzymes that are Ca-binding and/or Ca-activated including, but not limited to transglutaminases, enzymes which are Ca-binding and Ca-activated proteins. Secondary changes can occur in suitable conditions, for example, incubating at extended times adequate for the formation of cross linked CaBP-HA complexes wherein said secondary changes create neoepitopes on protein complexes created during primary binding. This process occurs in the preparation of a nanobacteria/calcifying nano-particle (NB/CNP) culture wherein transglutaminase initiates crosslinking in NB/CNPs. Commonly assigned U.S. Pat. No. 5,135,851, incorporated herein by reference, describes a method for the culture of NB/CNPs.

CaBP proteins bound to hydroxy apatite substrates form a CaBP-HA complex and that said CaBPs undergo a specific conformational change making these proteins appear antigenic thereby initiating an immune response in the body of a mammal. The formation of this mineral/protein complex and the antibodies created against the CaBP-HA by the host mammal provide for a means of detecting, analyzing, and assessing said antibodies and therefore the risk of, or disposition to disease or conditions.

The detection of anti-CaBP-HA complex antibodies provides a unique and novel diagnostic and prognostic tool for determining risk of disease as well as for determining a “cured” status in the patient. For instance, in study conducted by Kajander et al., 13 patients were treated with anti-CNP therapy consisting of 400 mg etidronate and 500 mg tetracycline daily for one week, thereafter half doses for 3 months. Antibodies to anti-CaBP-HA complexes were measured using a serum ELISA method during three months of therapy and followed three months after ending of therapy. The serum Antibody levels of 12 patients decreased, the mean reduction being 4.15-fold. Antibody levels of one patient were kept the same during 6-month period. The patient had taken calcium supplementation (i.e., Calcipos) against instructions. According to results, the antibody levels remained unchanged during 3-month period after ending of therapy. In chronic infections, such as peptic ulcers as caused by Helicobacter pylori infection in the stomach, eradication is measured and verified by observing a decrease in anti-Helicobacter pylori IgG titers. A 25% reduction in titres is considered a positive indicator for successful eradication of said pathogen. Therefore, based upon this analogy, anti-CaBP-HA complex antibody quantification can be a useful indicator for the successful therapy for NB/CNPs and the numerous diseases caused thereby.

Pathological calcification, or the unhealthy deposition of hydroxy apatite is found in numerous diseases, such as but not limited to Arteriosclerosis, Atherosclerosis, Coronary Heart Disease, Coronary Artery Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases. Lupus Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease) and Parkinson's Disease.

The disclosed method can involve the detection of one or more antibodies to CaBP-HA complex proteins that have undergone a conformational change. In the method of this invention, the antibodies are detected in the animal serum or plasma, cell culture samples, cerebrospinal fluid, urine, saliva, semen, amniotic fluid and cyst fluid of a mammal. In the preferred embodiment, the antibodies are tested in the blood, serum, or urine of a mammal.

Accordingly, the blood, serum, or urine may be assayed undiluted or diluted with an appropriate diluent (such as distilled water). with increasing sensitivities, dilution may be preferred (particularly when collection devices are used).

The CaBP-HA complex preparation will for convenience and preference be bound to a solid support. Suitable solid supports include a nitrocellulose membrane, glass or a polymer. Hydroxy apatite coatings and deposition thereof to numerous substrates is well known to those skilled in the art. Desirable polymers of use include, but are not limited to, cellulose, polyacrylamide, nylon, polystyrene, polyvinyl chloride or polypropylene. The solid supports may be in the form of strips, tubes, beads, discs or microplates, or any other surface suitable for conducting an immunoassay.

Antigen components may be produced via various routes including, but not limited to: 1) the use of synthetic hydroxy apatite between 0.020 and 200 μm in thickness that has been subjected to mammal blood or serum or purified proteins for an acceptable period of time under appropriate conditions in order to bind appropriate CaBP thereby allowing for the formation of CaBP-HA complex or 2) the use of NB/CNPs from a mammal wherein said biomineral, NB/CNP, is comprised of CaBP-HA complex.

Antibodies to CaBP-HA complex proteins are those antibodies to conformationally changed CaBP proteins and their complexes with other molecules. The following list is illustrative of many CaBPs but is not meant to limit the scope of this present invention. CaBP-HA complex conformationally changed proteins include, but are not limited to: proteins with a GLA-containing domain, clotting factor II, clotting factor VII, clotting factor IX, clotting factor X/Xa, tissue factor-clotting factor VIIa complex, prothrombinase complex (factor V, Xa, II), fragments of factor II, thrombin, prothrombin Fragment 1, matrix GLA-protein and osteocalcin, osteopontin, osteonectin, and proteins factor XIIIa, Fetuin A, calmodulin, Tissue Transglutaminase II, MMP-9, MMP-3, CD 42b, NF-kappa B, CD14, Fetuin B, CD40, myeloperoxidase, Fibronectin, tissue factor, human complement 5b-9, CRP , CD61, Kappa Light Chain, Macrophage L1 Protein, hsp 60, fibrillin-1, Beta-2-microglobulin, CD 18, laminin, antitrypsin, Notch-1, BSA, LPS-binding protein (LBP), PTX3, complement C5, fibrin/fibrinogen, D-Dimer, factor V, antichymotrypsin, Annexin V, vitronectin, thrombin, Troponin T, vimentin, tropomyosin, Human Serum Albumin, Troponin I cardiac, Apo A1, MHC class I, Amyloid P protein, sCD40 L, Kallikreins, ATIII, Factor VIII, Heparan Sulphate, Factor XI, c-jun, Fra-2, Fra-1, Jun B, P-c-Jun, Transglutaminase3, alpha fetoprotein, Prostate Specific Antigen (PSA), erbB2, VEGF, alpha synuclein, and other molecules interacting and binding to such complexes, such as LPS and its component Lipid A, Thomsen-Friedenreich antigen, and modifications of proteins such as isopeptide bonds made by transglutaminase.

Health risk is diagnosed by means of the invention by detecting the formation of a complex between the mammalian antibody and a second antibody, which may comprise anti-human or anti-specific species of animal IgG, IgM, IgA, IgE antibody (or mixtures thereof) in a blood, serum, or urine sample and anti-CaBP-HA antibodies. Some form of detection means is therefore necessary to identify the presence (or, if required, amount) of the antibody-antigen complex.

The detection means may be a second antibody, conjugated with a reporter molecule, and which is specific for at least one CaBP-HA antibody found in the mammalian fluid. A “reporter molecule” is a molecule or group which, by its chemical nature, has an analytically identifiable characteristic or provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e., radioisotopes). In the case of an enzyme immunoassay, an enzyme is conjugated to the second antibody, generally by means of glutaraldehyde or periodate. As will be readily recognized, however, a wide variety of different conjugation techniques exist, which are readily available to those skilled in the art. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable colour change. Substrates can be soluble or insoluble, depending upon the chosen application. For example, 5-bromo-4-chloro-3-indolyl phosphate/nitroblue tetrazolium is suitable for use with alkaline phosphatase conjugates; for peroxidase conjugates, 1,2-phenylenediamine-5-aminosalicylic acid, 3,3,5,5-tetramethylbenzidine, tolidine or dianisidine are commonly used. It is also possible to employ fluorogenic substrates, which yield a fluorescent product, rather than the chromogenic substrates noted above. Examples of fluorogenic substrates are 3-p-hydroxyphenylpropionic acid (HPPA) and dihydrotetramethylrosamine, examples of fluorogenic labels are fluorescein and rhodamine. When activated by illumination with light of a particular wavelength, the fluorochrome-labelled antibody absorbs the light energy, inducing a state of excitability in the molecule, followed by emission of the light at a characteristic colour which is usually visually detectable with a light microscope. Immunofluorescence and EIA techniques are both well established in the art and are particularly preferred for the present method. However, other reporter molecules, such as radioisotope, chemiluminescent, and bioluminescent molecules and/or dyes and other chromogenic substances, may also be employed.

The detection and measurement of anti-CaBP-HA antibodies can be useful for the diagnosis of diseases.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention. In the drawings:

FIG. 1 illustrates a histogram of numerous proteins as found on NB/CNP particles;

FIG. 2 illustrates data from a SAPIA test indicating that NB/CNPs contain conformationally changed proteins;

FIG. 3 illustrates a proteomic analysis of proteins on apatite particles briefly exposed to fetal bovine serum;

FIG. 4 illustrates the anti-NB/CNP antibody levels following the treatment protocol of example 4;

FIGS. 5 illustrates ELISA data for anti-Human CaBP-HA complex antibodies correlating to anti-Human Prothrombin F1-HA complex antibodies;

FIG. 6 illustrates Antibody reactivity against CaBP-HA complexes; and

FIG. 7 illustrates anti-CaBP-HA complex antibodies present in persons with diseases associated with pathologic calcification.

### DETAILED DESCRIPTION OF THE PREFERRED EMBODIMENTS

The disclosed method and compositions may be understood more readily by reference to the following detailing description of particular embodiments and the examples included therein and to the figures and their previous and following description.

Disclosed are methods and compositions for detection, analyzing, and assessing the presence of CaBP-HA complexes. The disclosed methods and compositions generally involve detecting one or more antibodies to CaBP-HA complex present in a mammals biological fluid.

The immunoassay test kit in accordance with an embodiment of the invention incorporates several components, including a preparation of a second antibody, which may comprise anti-human or anti-specific species of animal IgG, IgM, IgA, IgE or a mixture thereof, the conjugation of the second antibody to a probe, preparation of antigen, preparation of antigen coated microtiter plates or vertical test strips, preparation of the reagents and the immunoassay. Immunoassays useful in the practice of the invention include but are not limited to assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, quantum dot assays, and electrochemiluminescence or other technique known to those skilled in the art. Thus, by way of example, the invention will be described in relation to ELISA. However, it should be understood that the following embodiments relate to any immunoassay incorporating the composition of the invention, and is not limited to any particular type of immunoassay.

Accordingly, the preparation of the second antibody, which may comprise anti-human or anti-specific species of animal IgG, IgM, IgA, IgE or a mixture thereof, may be made by any method which is well known to persons skilled in this art.

Anti-human or anti-specific species of animal IgG, IgM, IgA, or IgE antibodies recognizing anti-CaBP-HA complex antibodies can bind anti-CaBP-HA complex antibodies. They can be utilized to immobilize anti-CaBP-HA complex antibodies or to remove anti-CaBP-HA complex antibodies from a biological sample in the purposes of elimination, assay and purification. Antibodies can be used in many ways to construct an immunoassay for anti-CaBP-HA antibodies. Also, the antibodies may find use in elimination of anti-CaBP-HA antibodies from cell cultures or from animals or from humans. Further, the antibodies may be applicable for purification of products derived from cell cultures, animals or humans. These may include blood, serum and their products, or cells and organs, or in vitro cultured products including cells.

Notably, because the presence of CaBP-HA complexes and conformationally changed proteins thereon, have been implicated in the pathogenesis of intracellular and extracellular calcification and calcification-related diseases, the anti-human or anti-specific species of animal IgG, IgM, IgA, or IgE antibodies can be used to detect and quantify the presence of anti-CaBP-HA complex antibodies and, in fact, the presence of anti-NB/CNPs antibodies in a specimen. This detection and quantification can, in turn, be used for diagnostic and prognostic purposes by associating the level of anti-CaBP-HA complex antibodies with levels of intracellular and extracellular calcification or for monitoring the response to treatment or therapy.

Following the preparation of the anti-human or anti-specific species of animal IgG, IgM, IgA, or IgE antibody, the next step may involve the conjugation of the antibody to the probe. In one embodiment of the invention, this may be accomplished by first dialyzing the antibody solution in approximately 0.01 M sodium carbonate buffer at pH 9.0 overnight. Then, in accordance with practices known in the art, the enzymatically-labeled probe should be prepared. One embodiment of preparing the probe entails first mixing, in separate containers, solutions of horseradish peroxidase (HRP) and NaIO₄. For example, in one container, about 2 to 10 mg of HRP is added to about 1 mL of water. In another container, about 21.4 mg NaIO₄ is added to about to 1 mL water. Then the NaIO₄ solution and the HRP solution can be combined by pipetting approximately 100 micro-liters of the NaIO₄ into the HRP mixture. That mixture should then stand at room temperature for approximately 10 minutes, during which time the color will change to dark green. In the meanwhile, according to this embodiment of preparing the antibody-conjugate probe, the probe is dialyzed against 5 mM sodium acetate buffer at pH 4.0 overnight. The next day, about 0.2 M sodium carbonate buffer, at pH 9.5, is added to the Na Acetate buffer containing the activated HRP. This buffer solution is then mixed with the antibody solution and the resulting antibody-buffer mixture is incubated twice. The first incubation of the antibody-buffer mixture should take place for approximately two hours at room temperature. At the close of those two hours, about 100 micro-liters of 0.1 M NaBH₄ in water should be added and then, the second incubated should occur, in which the resulting mixture is incubated at 4° C. for two additional hours. Finally, the resulting mixture is placed in a dialysis tube and dialyzed against PBS overnight.

In another embodiment, about 5 mg/mL monoclonal or polyclonal antibody solution in 0.1 M phosphate buffer at pH 6.8 (PBS) is dialyzed overnight at approximately 4° C. Then, about 0.5 mg of the dialyzed monoclonal or polyclonal antibody is added to about 1.5 mg of alkaline phosphatase (immunoassay grade, Boehringer Mannheim, Indianapolis, Ind.) in about 10 mL of 10 mM PBS. Once that mixture is prepared, 80 micro-liters of 25% glutaraldehyde is added and the solution is gently mixed to combine. The resulting solution should then stand at room temperature for approximately 2 hours. After 2 hours, the reaction is stopped by adding, in an approximately equivalent volume (10 mL), PBSLE (10 mM PBS containing 100 mM lysine and 100 mM ethanolamine). The solution must then be desalted. According to this embodiment, the solution is desalted with Sephadex G25 column in PBSN (10 mM PBS with 0.05 M NaN₃). After the alkaline phosphatase-antibody conjugate is desalted, 20 mL of the conjugate should be mixed with 40 mL of blocking buffer (0.17 M borate buffer containing 2.5 mM MgCl₂, 0.05% Tween 20, 1 mM EDTA, 0.25% BSA and 0.05% NaN₃). Finally, 60 mL of the conjugates are filtered through a low-protein binding filter, Millex HV 0.45 micro-m (Millipore Corp. Bedford, Mass.), for sterilization. Once filtered and sterilized, the conjugate can be stored at 4° C.

In another embodiment, a substrate solution manufactured by MOSS, Pasadena, may be utilized to function as substrate for the probe, as will be known by those skilled in the art.

The next process involves the antigen preparation for immunoassay. There are two discrete methods and starting compositions for the preparation of the antigen. 1) The use of sonicated nanobacteria/calcifying nano-particles as collected from the body of a mammal or 2) commercially available or synthetic calcium phosphate mineral hydroxy apatite that is subjected to mammalian blood or serum or to a purified protein or a mixture of proteins for a long enough time to allow for primary binding and, as desired secondary conformational changes of CaBPs contained in said blood or serum, or solution having added transglutaminase or other suitable enzyme.

The antigen culture method of culturing NB/CNP under conditions similar to tissue culture. Any standard tissue culture media is useful in the practice of the present invention. Cell or tissue culture media are generally used to culture mammalian cells. During culture, NB/CNPs become visible under light microscopy due to their multiplication, aggregation, secretion of biofilm and/or the thickening of their apatite cell envelope. Their detection is aided by providing nonviable or “killed” NB/CNPs for comparison as a control. Furthermore, a modification of the Hoechst staining method has been developed to verify that the particles contain stainable nucleic acids although much less than common bacteria, which could also be contaminants in the culture. This method is disclosed in commonly assigned U.S. Pat. No. 5,135,851 which is incorporated herein by reference.

One embodiment of the present invention involves a synthetic medium suitable for the replication of NB/CNPs by fulfilling their growth requirements. The liquid medium may comprise a standard tissue culture medium known as RPMI 1640 or DMEM. This medium is a standardized composition of amino acids, salts, etc. which can be obtained from Gibco (Uxbridge, Middlesex, U.K.).

The components of the culture medium should be dissolved in essentially sterile water. The quality of water used is extremely important, since water can contain cytotoxic impurities for the unidentified agents. Care must be taken to avoid water as a source of contamination. Tap water, deionized water or sterile water for injection, for instance, may all be adequate if their sterility is checked in advance.

When it is desired to use NB/CNPs as the antigen of the present invention, The culture media can also be solidified using agar or agarose. However, once again, agar or agarose may contain cytotoxic impurities. Growth is also optionally stimulated by the addition of nucleotide precursors and supplements such as L or D,L-selenomethionine. Thus, the medium is preferably supplemented with a mixture (50-100× concentrate) prepared separately from D,L-selenomethionine, adenosine, thymidine, uracil, guanine and cytosine all of which can be obtained from Sigma Chemical Co., St. Louis, Mo. For example, the 100×-concentrate contains 10 mM DL-selenomethionine and 1 mM by each of the following compounds: adenosine, thymidine, uracil, guanine and cytosine, dissolved in a solvent. The final medium is prepared by adding 1 mL of the dissolved 100× concentrate to 99 mL of the basal medium. In the preparation of the basal medium and of the supplement, deionized or distilled water is utilized. Standard procedures in utilizing pharmaceutical grade components and biologically sterilized equipment are followed.

NB/CNP cultures are scrubbed gently with a cell scraper. Cultures are transferred in centrifuge tubes. Tubes are centrifuged at approximately 15 000 g for 45 minutes with an ultra centrifuge. Pellets are washed with sterile PBS and pooled into sterile eppendorf tubes. Approximately 50 μL volume of colloidal nanomaterial-mineral complex is suspended in 10 mL PBS and sonicated 2×10 second (at 10 second intervals off). Sonication is repeated until turbidity value reaches a plateau. During sonication, temperature is controlled by cooling with ice, temperature reaches a maximum is 60° C. The suspension is diluted to 400 mL with PBS. Turbidity value of the solution is acceptable at between 5-20 NTU. This solution, the coating solution, is then pipetted 100 μl/well on ELISA multi-well plates. Plates are incubated between +2 and +8° C. overnight without shaking. Plates are washed once with TBS-Tween. Plates are then blocked by pipetting a blocking solution containing Tween, 300 μl /well. Plates are then incubated 2 hours at RT or overnight at between +2 and +8° C. (without shaking). Plates stored as wet plates are prepared by washing the plates once with TBS-Tween, and adding storage solution with a suitable protective agent (NaN₃) 150 μL/well. Plates are sealed with tape and stored in humidified boxes. Alternatively, dry plates are prepared by washing the plates once with TBS-Tween. Plates are thereafter saturated by adding a saturating solution 100 μl/well containing sucrose, sorbitol or other suitable sugar, and incubated on plate shaker for 5 minutes. Plates are emptied by tapping them dry against paper. Plates are put to drying oven at +30° C. (circulating air) with silica gel and incubated for 2½ hours (until dry). Plates are taken out of the oven and stabilized at RT. Plates are then packed into the aluminium bags with desiccant bags (Desi Pak®)(11 g/plate) and plate package is heat sealed. Plates are stored at between +2 and +8° C.

Mineralization of NB/CNPs, which is particularly pronounced in older cultures (>1 month), is known to interfere with the assay. This can be avoided by using a calcium chelator, at a final concentration of 30 mM, in the diluted sample. In accordance with embodiments of the invention, the calcium chelators may include one or more of Ethylenediaminetetraacetic acid (EDTA), Ethyleneglycoltetraacetic acid (EGTA), Diethylenetriaminepentaacetate (DTPA), Hydroxyethylethylenediaminetriacetic acid (HEEDTA), Diaminocyclohexanetetraacetic acid (CDTA), 1,2-Bis(2-aminophenoxy)ethane-N,N,N′,N′-tetraacetic acid (BAPTA), and pharmaceutically acceptable salts thereof. The addition of a calcium chelator exposes all of the available antigen yet has no effect on the background. The signals from young (<1 month) cultures of NB/CNPs increase 2-4 fold as a result of the calcium chelator treatment and the signals from serum-free cultures increase more than ten-fold as a result of the calcium chelator treatment. The calcium chelator has no effect on the signals obtained from non-cultured NB/CNPs. Thus, in accordance with this invention, the kits and methods disclosed herein may comprise addition of a calcium chelator to the assay buffer used to dilute the samples when cultivated NB/CNPs are tested. In one embodiment of this invention, 500 mM EDTA in water, pH adjusted to approximately 7.5 with NaOH, is used.

Alternatively, serum, serum proteins, or purified proteins from human or bovine sources can be bound on synthetic hydroxy apatite (HA) surfaces by exposing such surfaces to protein solutions for one minute to one month. During this time, proteins will adsorb on the HA surface and acquire their conformation typical for interaction with HA surface. Furthermore, neoepitopes may be created by covalent modifications, including cross-linking, e.g. by enzymes like transglutaminase, that are naturally present in serum, or can be added to the protein solution. After the coating procedures the material must be washed and blocked before use in immunoassay as described above. The method can use saturated calcium and phosphate solutions to make apatite surface on ELISA plate or other suitable devices at low temperature as shown in Example 4. Furthermore, other methods for preparing synthetic HA coating can be applied to materials suitable for such processes that are well known to those in the art. Apatite coating is transparent to visible light and therefore is compatible with optical reading and other optical or light scattering analytical devices and protocols well know to those in the art.

Specifically, human auto-antibodies against conformationally changed proteins bound on hydroxyapatite were measured using Synthetic Apatite Acting as Substratum. Synthetic apatite can be made using several published procedures. To coat plastic surfaces, the synthetic apatite was prepared according to Poser and Price [J. W. Poser, P. A. Price, J Biol Chem 254, 43 (1979)] using sterile solutions. An alternative is to use highly heated calcium phosphate spray, which may be used for coating on metal and ceramic type surfaces suitable for that. 100 mL/well of hydroxyapatite solution was pipetted onto 96-well polystyrene plates (Nunc). Plates were dried in circulating air oven at temperature +37° C. overnight. Purified protein, human Prothrombin Fragment 1.2 (USBiological), was diluted with PBS to a concentration of 10 mg/mL, and the solution was added 100 μl/apatite-coated well. Plates were incubated at +4° C. overnight. The next day, the protein solution was removed and the plate was washed once with TBS-Tween, and blocked for 2 hours at room temperature with Tween-containing solution The blocking solution was removed and plates were prepared for storage as wet plates or dry plates. Wet plates were prepared by adding storage solution (NaN3/Proclin-PBS) 300 mL/well, and sealing the plates with tape. For longer shelf-life, the plates were dried after blocking as above and saturating with a solution containing sucrose or other sugars for 5 minutes. The saturating solution was removed and plates were dried in oven at +30° C. for 2½ hours. After drying, plates were sealed with tape or placed into aluminum bags together with an activated desiccant bag to control moisture level. Plates were washed prior to use with TBS-Tween. Serum or plasma test samples were diluted 1:500 with Tween-containing Assay Buffer and pipetted 100 mL/well in duplicates. Assay can be quantitated with dilution series made with a positive control sample. Samples were incubated 1 hour at room temperature with moderate shaking and after incubation, plates were washed 4 times with TBS-Tween. Secondary antibody HRP-anti-human IgG (Zymed) was diluted 1:4000 into diluent solution containing Tween of which 100 mL was added to all wells. Plates were incubated 1 hour at room temperature with moderate shaking. Plates were washed four times with TBS-Tween and 100 mL substrate solution TMB (Moss) was added to all wells. Plates were then incubated while being protected from light and absorbance was read at 630 nm. The reaction was able to be stopped with H₂SO₄ and absorbance read at 405 nm or 450 nm, when necessary.

Hydroxy apatite is, as disclosed previously, able to be precipitated out of a saturated solution of calcium and phosphate and thereby adhered to a suitable substrate. HA is also able to be formed into solid particles such as spheres or the like via condensation and/or solvent extraction mechanisms such as spray drying or fluid bed drying. The form or source of HA is not limiting to the object of this invention and it is to be known that any HA surface is an appropriate substrate for the formation of the CaBP-HA complex.

The next process involves preparation of the reagents to be used with the immunoassay kit disclosed in the present invention. In one embodiment of the invention, the reagents may include reagents to be used with 96-well microtiter plates and will comprise standards for target values, assay buffer (50 mL), secondary antibody concentrate (1.1 mL), secondary antibody diluent (12 mL), wash buffer concentrate (25×) (40 mL), substrate solution (as described above, to be used as the probe) (13 mL), stop solution (13 mL), microtitration plate (coated as described above), and a positive control, and as a negative control, assay buffer will be used.

Detection of the anti-CaBP-HA complex antibodies in a test sample can occur by a variety of methods. In one embodiment, an immunoassay is performed to identify anti-CaBP-HA complex antibodies in a specimen collected from an individual. The specimen is spun and the serum separated from the clot within two hours of collection. Samples can be stored at 2 to 8° C. for up to four days, and at −20 to −80° C. for at least one year. Repeated freezing and thawing should be avoided.

In another embodiment, the immunoassay is performed using fluorometric assay, chemiluminescent assay, or radioimmunoassay to detect and measure the anti-CaBP-HA complex antibody levels. In this embodiment, the second antibodies are preferably labeled, either directly or indirectly with a detectable label, such as a radioisotope or a detectable molecule or protein. Various types of labels and methods of conjugating the labels directly or indirectly to the polypeptides and antibodies are well known to those skilled in the art and include enzymes, radioisotopes, and fluorescent, luminescent and chromogenic substances including colored particles such as colloidal gold and latex beads. The following examples are labels are suitable in immunoassays including enzyme-linked immunosorbent assays (ELISA) and radioimmunoassay: (1) The antibodies may be conjugated to a radiolabel such as, but not restricted to, ³²P, ³H, ¹⁴C, ³⁵S, ¹²⁵I, or ¹³¹I. Detection of a label can be by methods such as scintillation counting, gamma ray spectrometry or autoradiography; (2) alternatively, bioluminescent labels, such as derivatives of firefly luciferin, may be used. The bioluminescent substance is covalently bound to the polypeptide by conventional methods, and the labeled polypeptide is detected when an enzyme, such as luciferase, catalyzes a reaction with ATP causing the bioluminescent molecule to emit photons of light; (3) or, fluorogens may also be used as labels. Examples of fluorogens include fluorescein and derivatives thereof, phycoerythrin, allo-phycocyanin, phycocyanin, rhodamine, and Texas Red. The fluorogens are generally detected by a fluorescence detector; (4) further still, the polypeptides and antibodies can alternatively be labeled with a chromogen to provide an enzyme or affinity label. For example, the antibody can be biotinylated so that it can be utilized in a biotin-avidin reaction, which may also be coupled to a label such as an enzyme or fluorogen; (5) alternatively; the antibody can be labeled with peroxidase, alkaline phosphatase or other enzymes giving a chromogenic or fluorogenic reaction upon addition of substrate. Additives such as 5-amino-2,3-dihydro-1,4-phthalazinedione (also known as Luminol.TM.) (Sigma Chemical Company, St. Louis, Mo.) and rate enhancers such as p-hydroxybiphenyl (also known as p-phenylphenol) (Sigma Chemical Company, St. Louis, Mo.) can be used to amplify enzymes such as horseradish peroxidase through a luminescent reaction, and luminogeneic or fluorogenic dioxetane derivatives of enzyme substrates can also be used; (6) In addition, the second antibody may be labeled with colloidal gold for use in immunoelectron microscopy or in lateral flow rapid test in accordance with methods well known to those skilled in the art. Such labels can be detected using enzyme-linked immunoassays (ELISA) or by detecting a color change with the aid of a spectrophotometer, refractometer or scanner. These and other methods of labeling antibodies and assay conjugates are well known to those skilled in the art. The above labels are listed for example only and are not intended to limit the scope of labels that are known in the art.

Immunogenic tests may be performed by various methods. The following Example 1 describes one method by which the ELISA test can be performed. The following Example 2 describes one method by which the quantification of anti- NB/CNP antibodies can be used to determine patient susceptibility to Coronary Artery Disease. Example 3 describes immune Response in humans against biogenic and artificially made CaBP-HA complexes. These examples are non-limiting and are offered only as illustrations of two of the many methods by which the immunogenic tests can be performed to detect anti- NB/CNP antibodies in the specimen and to employ such immunogenic tests for diagnostic and prognostic purposes.

In the Figures:

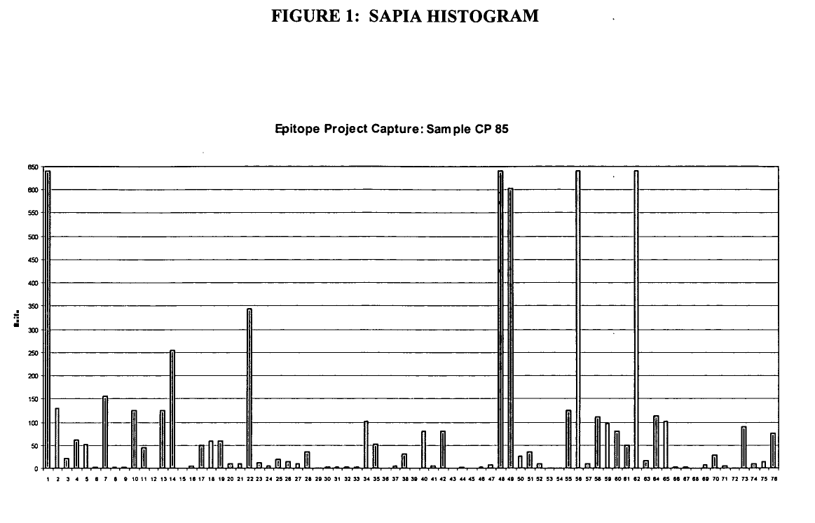

FIG. 1 is a histogram including, but not limiting, numerous proteins as found on NB/CNP particles. Surface Antigen Pattern Immunoassay (SAPIA) test was developed to detect presence of multiple proteins on CNPs. SAPIA plates were made by coating high binding polystyrene ELISA plates (Corning, USA) with antibodies against anti-calcification proteins and Gla clotting factors and control antibodies. SAPIA was controlled by using antibodies against human serum albumin, D-Dimer, NF-κB and fibronectin as these proteins were not expected to be specifically bound on particle surface. Monoclonal antibodies were diluted at a final concentration of 1 μg/ml with 1×PBS, pH 7.4,100 μL/well to ELISA plates and incubated at +4° C. overnight. Polyclonal antibodies were diluted to a concentration of 10 μg/ml and plates were coated as above. After coating procedure, plates were washed once with TBS-Tween 20 and blocked by adding TBS-Tween 20 300 μL/well and incubated 2 h at room temperature. Thereafter, blocking solution was removed and storage solution 0.05% NaN3-TBS was added 200 μL/well, and the plates were stored sealed with tape in a refrigerator. Before use, storage solution was removed and plates were washed once with TBS-Tween. 50 μL/well of Assay Buffer (0.05 M Tris, 0.15 M NaCl, 0,05% Proclin 300, pH 7.5 with 1% mouse serum) was added in duplicates and 50 μL/well serum sample was added. Plate was sealed with tape and incubated 1 h at room temperature with moderate shaking. Plates were washed four times using TBS-Tween and detection antibody HRP-8D10 (Nanobac Oy) was added 100 μL/well. Plates were incubated 1 h at room temperature with moderate shaking. Plates were washed four times using TBS-Tween and TMB substrate (Moss Inc., Pasadena, Md. 21123) was added 100 μL/well. Plates were sealed and protected from light with foil and incubated 20 min at room temperature with moderate shaking. Absorbance at 630 nm was read with microplate reader (Biohit BP 808). Blank values were subtracted and unit values were calculated from standard curve of Nanocapture ELISA test using TableCurve 2D program (Systat, Point Richmond, Calif.). Pearson Correlation Coefficients (N=16, Prob>|r| under H0: Rho=0) were calculated. Anti-calcification proteins and coagulation Gla factors were found to be present in CNPs together with several other proteins.

SAPIA method was first tested using plasma and serum samples from 8 persons. Human plasma and serum samples were first tested to determine if they are comparable samples. Note that the results of positive sample shown are exceptionally high for clotting factors. Based on the results obtained with these 8 samples, both serum and plasma samples could be used to test the specific parameters. Thereafter, a random panel (n=16, each sample was combined from 1-5 human serum samples of similar CNP unit values to make up the necessary volume needed to run many tests, done in duplicate) with ELISA results ranging from 0-640 units was selected for the SAPIA test.

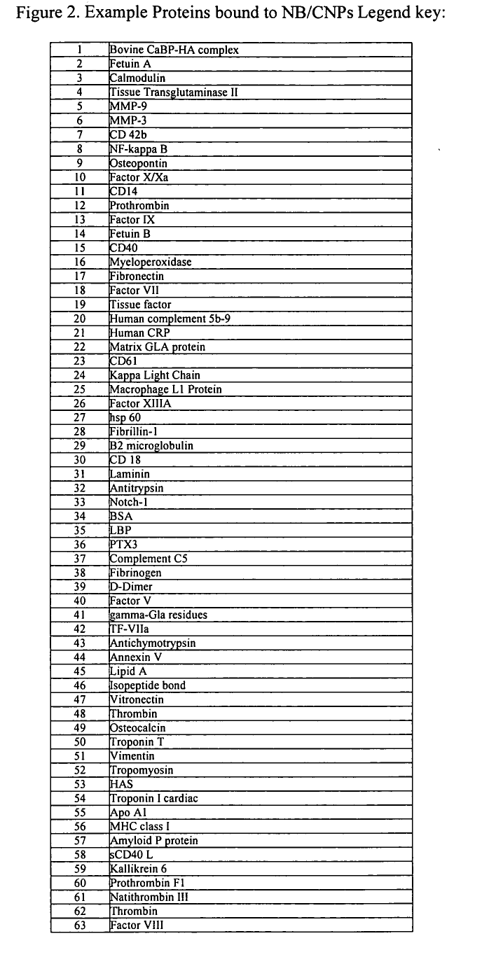

FIG. 2 illustrates data from a SAPIA test indicating that NB/CNPs contain, within or on the hydroxy apatite coating, conformationally changed proteins including, but not limited to: proteins with a GLA-containing domain, clotting factor II, clotting factor VII, clotting factor IX, clotting factor X/Xa, tissue factor-clotting factor VIIa complex, prothrombinase complex (factor V, Xa, II), fragments of factor II, thrombin, prothrombin Fragment 1, matrix GLA-protein and osteocalcin, osteopontin, osteonectin, and proteins factor XIIIa,Fetuin A, calmodulin, Tissue Transglutaminase II, MMP-9, MMP-3, CD 42b, NF-kappa B, CD14, Fetuin B, CD40, myeloperoxidase, Fibronectin, tissue factor, human complement 5b-9, CRP , CD61, Kappa Light Chain, Macrophage L1 Protein, hsp 60, fibrillin-1, Beeta-2-microglobulin, CD 18, laminin, antitrypsin, Notch-1, BSA, LPS-binding protein (LBP), PTX3, complement C5, fibrin/fibrinogen, D-Dimer, factor V, antichymotrypsin, Annexin V, vitronectin, thrombin, Troponin T, vimentin, tropomyosin, Human Serum Albumin, Troponin I cardiac, Apo A1, MHC class I, Amyloid P protein, sCD40 L, Kallikreins, ATIII, Factor VIII, Heparan Sulphate, Factor XI, c-jun, Fra-2, Fra-1, Jun B, P-c-Jun, Transglutaminase3, alpha fetoprotein, Prostate Specific Antigen (PSA), erbB2, VEGF, alpha synuclein, and other molecules interacting and binding to such complexes, such as LPS and its component Lipid A, Thomsen-Friedenreich antigen, and modifications of proteins such as isopeptide bond made by transglutaminase.

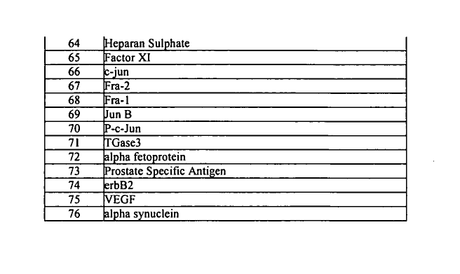

FIG. 3 is a table illustrating a proteomic analysis of proteins on apatite particles briefly exposed to fetal bovine serum shows the attachment of various CaBPs to a hydroxy apatite subtrate after incubation period. Protein-free apatite particles in DMEM (Gibco) without any additives were suspended into 10% FBS-DMEM and were immediately centrifuged at 14 000 rpm, 30 min at +4° C. The pellet was washed two times by suspending with sterile PBS followed by centrifugation at 13 200 rpm, 20 min at room temperature. The pellet was frozen prior to analysis. Proteomics analysis was provided by Protana, Montreal, Canada. The SDS-boiled samples were subjected to 1D SDS-PAGE under reducing conditions. Protein bands were detected by Coomassie staining, excised and processed following standard procedures including: 1) the proteins in the gel plug were reduced with DTT; 2) the free cysteine residues were alkylated with iodoacetamide; 3) the proteins were digested with the endoprotease trypsin; and 4) the peptides produced were extracted in neutral, acidic and basic conditions.

The peptide mixtures were separated by C18 reverse phase chromatography into a Thermo-Finnigan LTQ-FT ion trap/FTICR hybrid mass spectrometer coupled with a nano-spray interface. The mass spectrometer was operated in data-dependent mode to obtain tandem (ms/ms) spectra of each peptide above an intensity threshold as it emerged from the chromatography column. The raw data files were processed using LCQ-DTA to generate peak lists of the tandem spectra. The processed data was searched with Mascot (Matrix Sciences, London UK) using the NCBI non-redundant database. The Mascot results were curated by mass spectrometry scientists to correlate the results with the raw data.

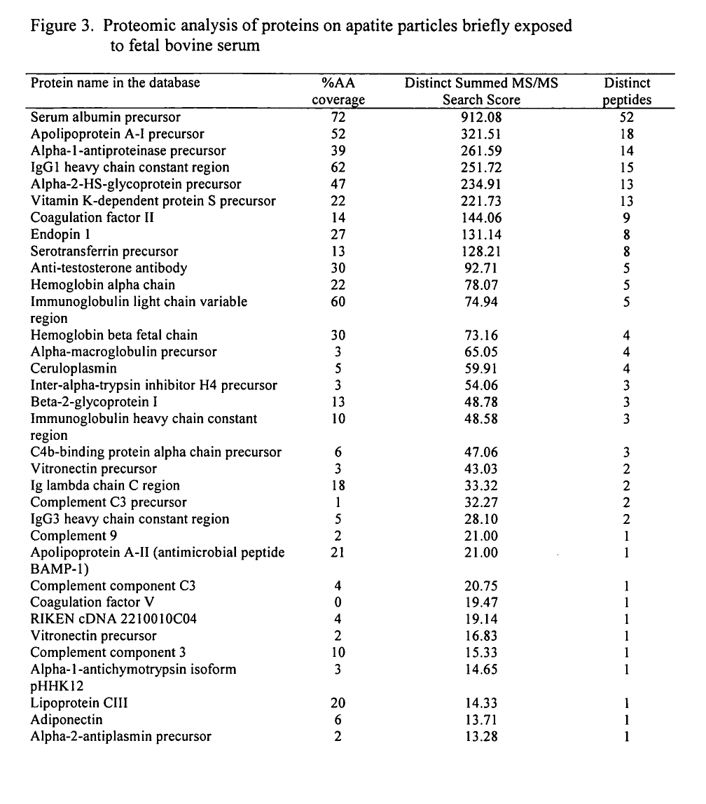

FIG. 4 shows anti-NB/CNP antibody levels following the treatment protocol of example 4. wherein 13 patients were administered etidronate and tetracycline for a 3 month period. The chart shows a drastic reduction in the antibody levels of the patients during the treatment and throughout the follow up indicating that antibody titre levels using the novel antigen complex is a useful diagnostic tool for the quantification of response to treatment

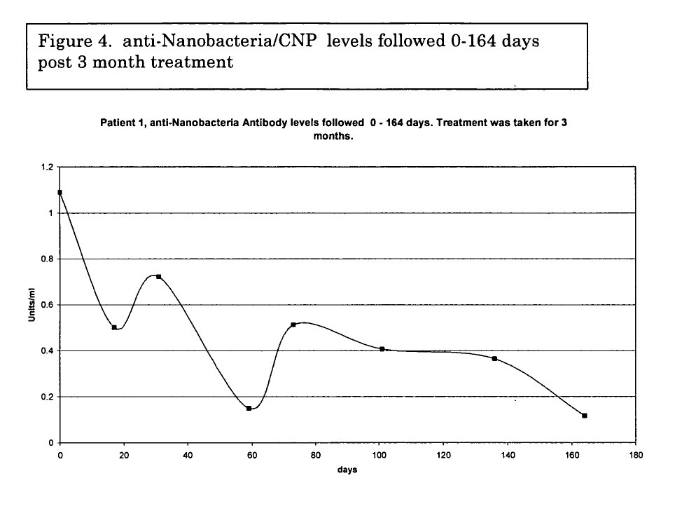

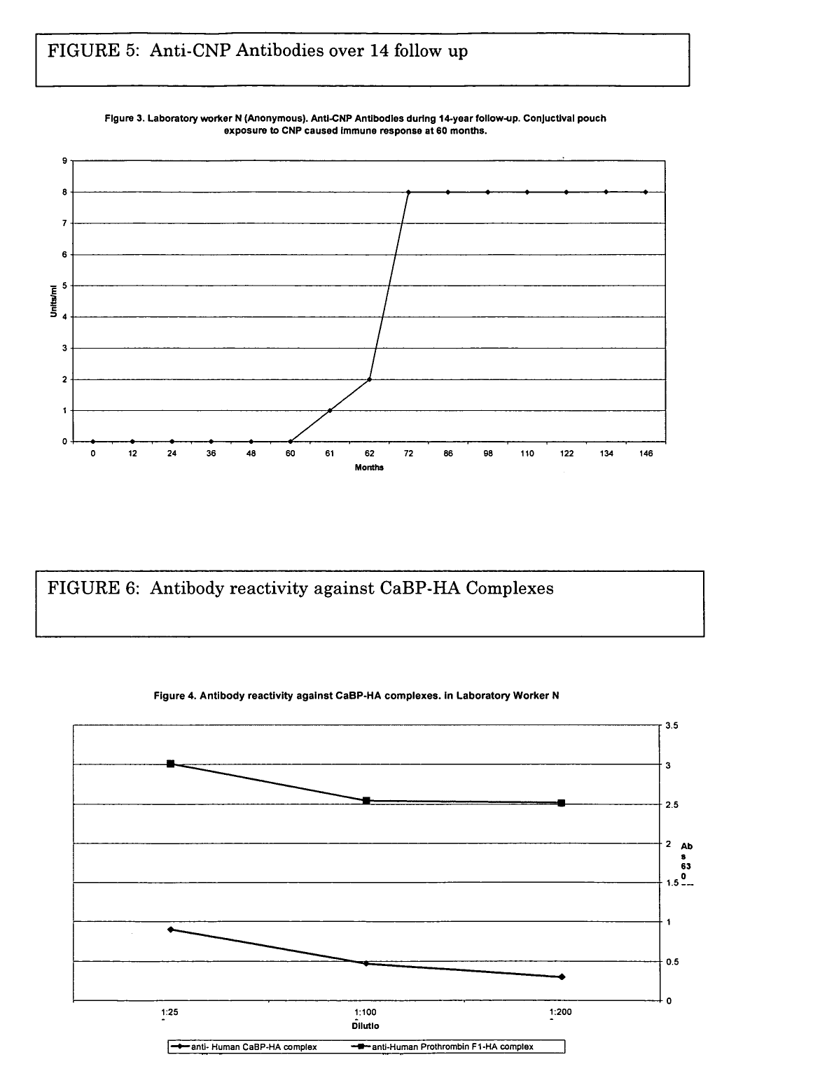

FIGS. 5 and 6 are line graphs showing antibody levels in a laboratory worker who was exposed to a pure culture of NB/CNPs at 60 month point of follow-up. The route of exposure was via the conjunctival pouch (eye). FIG. 4 illustrates a rapid increase in the anti-NB/CNP antibodies at 60-63 months. Post 72 months following exposure, and with no attempt at treatment, antibody levels stabilized and have remained static since. FIG. 5 illustrates ELISA data for anti-Human CaBP-HA complex antibodies as correlating to anti-Human Prothrombin F1-HA complex antibodies. This chart illustrates the efficacy of the use of anti-CaBP-HA complex antibody testing, exampled by using human biogenic CaBP-HA complex and synthetically made Human Prothrombin Fragment 1-HA complex as antigens, in a subject that is known to have NB/CNP infection. The rate of anti-Human CaBP-HA complex antibody follows that of the anti-Human Prothrombin F1-HA complex antibodies throughout dilution.

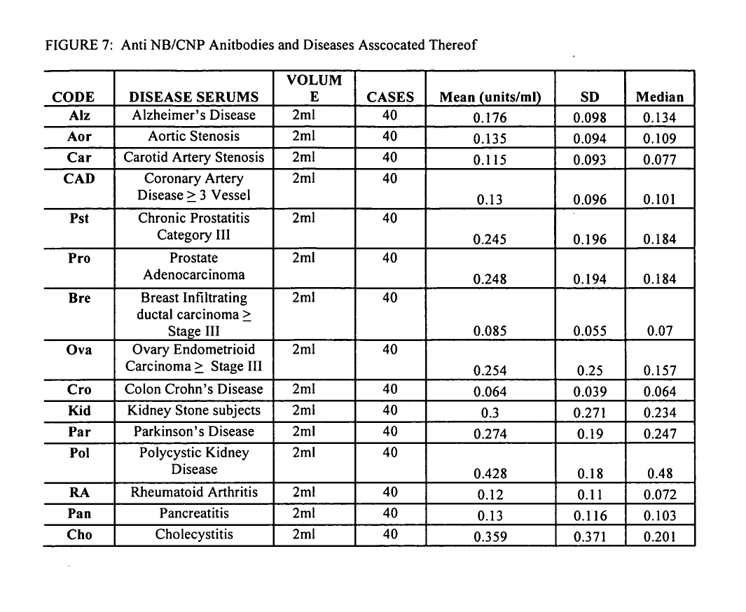

FIG. 7 illustrates anti-CaBP-HA complex antibodies present in persons with diseases associated with pathologic calcification; figure corresponds to Example 5, wherein 600 serum samples were acquired commercially from Clinomic BioScience, Watervliet, N.Y. Serum antibodies were detectable in all disease groups except for the serum samples collected from patients with Crohn's disease. Mean anti-NB/CNP antibody values were statistically greater for all disease groups versus Crohn's Disease. These data indicate that levels of anti-NB/CNP antibodies are associated with numerous disease including, but not limited to, Alzheimers, Aortic Stenosis, Carotid Artery Stenosis, Coronary Artery Disease, Chronic Prostatitis, Prostate Cancer, Breast Cancer, Ovarian Cancer, Kidney Stone Disease, Parkinson's Disease, Polycystic Kidney Stone Disease, Rheumatoid Arthritis, Pancreatitis, and Cholecystitis and may be an early warning sign of disease.

### EXAMPLE 1

### ELISA Test

PROCEDURE: Blood or serum is collected from a mammal via appropriate and well known protocols established in the art. Freeze/thaw cycles are to be avoided. Prior to testing, the reagents must be prepared by appropriate dilution and then allowed to come to room temperature, typically by allowing said reagents to sit at room temperature for approximately 15 minutes. Reagents should never be interchanged between test kits with different lot numbers.

The microplate used in the assay is removed from the bag and allowed to come to room temperature prior to use. Once the desired number of test strips have been removed, immediately reseal the bag and store at 2-8° C. to maintain plate integrity.

The standards and controls need to be reconstituted by adding 1 mL of distilled or deionized water to each vial. Replace the stopped and allow the material to stand for at least 15 minutes at room temperature. Mix the standards and control well before use. Subsequent to reconstitution, the stability of the standards and control is one day at room temperature. They can be frozen but must be used within one month. Do not freeze thaw more than one time. The wash buffer and conjugate supplied with the kit are diluted appropriate with distilled or deionized water and mixed. Patient serum is diluted 2μL/mL with incubation buffer and is allowed to sit for at least 15 minutes at room temperature.

Prior to the assay, wash each test strip that will be used only, once, with wash solution. Pipette 100 μL of each standard and controls and diluted samples in appropriate wells. Incubate for 60 minutes at room temperature (22±2° C.) on a plate shaker at 100 rpm±10. Using good laboratory techniques, carefully and manually wash avoiding contamination between the wells. Decant the well contents by inverting the plate over a container and without re-inverting, blot the plate against absorbing paper. Wash each well four times with 300 μL of wash solution. At the last wash, decant completely the wash solution by tapping the plate against absorbing paper until no trace of liquid is visible on the paper. Then, add 100 μL diluted conjugate to each well. Incubate for 60 minutes at room temperature (22±2° C.) on a plate shaker at 100 rpm±10. Wash again manually, taking precautions to avoid cross-contamination between wells. Decant the well contents by inverting the plate over a container and without re-inverting, blot the plate against absorbing paper. Wash each well four times with 300 μL of wash solution. At the last wash, decant completely the wash solution by tapping the plate against absorbing paper until no trace of liquid is visible on the paper. Pipette 100 μL of TMB Substrate in each well. At this juncture of the procedure, protect from light. Incubate for 20 min. at room temperature (22±2° C.) on a plate shaker at 100 rpm±10. Pipette 100 μL of Stop solution in each well. Read the absorbance at 450 nm. Read the absorbance immediately.

CALCULATIONS: Data is examined for acceptance consistency with quality control guidelines. Aberrant values may be rejected. The absorbance value of the standard zero should not exceed 0.3. However, it is an indication of careless washing and the assay must be repeated. For each standard, control and unknown sample, the optical density values are averaged (if there is duplicate). Subtract the means of the absorbance values of the zero standard from mean absorbance values of other standards, control and samples. On millimeter paper using the ordinate for the optical density and the abscissa for the standard concentrations (Units/mL), a smooth standard curve is plotted. Alternatively, 4-parameter curve fit programs can be used. The values of the control and unknown samples are read directly from the standard curve. The sample dilution factor (2 μL/mL or 1/500) and the sample volume factor (100 μL or 1/10) are included in the standard curve concentrations. Therefore, the values of the unknowns samples are read directly from the standard curve and are not multiplied by these factors.

INTERPRETATION OF RESULTS: Expected value is <0.2 Units/mL (negative result). Positive result (>0.2 Units/mL) indicates presence of antibodies against CaBP-HA complexes. Positive result with serum or plasma specimens is very common in diseases with arterial calcification or other forms of pathological calcification.

### EXAMPLE 2

### Correlation Between Anti-NB/CNP Antibodies & Coronary Artery Disease

Studies have suggested that coronary calcification is related to the risk of coronary artery disease (CAD). The present example examined whether antibodies against NB/CNPs are associated with levels of coronary calcification that appear to reflect preclinical CAD.

Serum specimens from 201 healthy asymptomatic subjects (52% male, mean age 56.6 years) undergoing electron beam computed tomographic imaging were used to measure antibodies to NBICNPs, to other infectious pathogens and to CRP levels. High calcification score was defmed cutoff value for defining a level of coronary calcification≧75^{th} percentile, and high antibody level for defining a NB/CNP antibody level≧II tertiles.

Serum antibodies against NB/CNPs were detectable in 94% of study subjects. Mean antibody value was 1.07 units, and antibody tertile values were 0-0.21, 0.22-0.73 and 0.74-19.26 units, respectively. There was 31% of individuals with high calcification scores in the high antibody titer group, compared with 16% in the lowest titer group (P=0.024). The association of increased levels of NB/CNP antibodies with high coronary calcification scores was independent of CAD risk factors, including age, male sex, smoking, diabetes, hypertension, hypercholesterolemia and family history of CAD (adjusted odds ratio OR of 3.15 with 95% confidence limit 1.39 to 7.15). However, no association was found either between coronary calcification and CRP level, or between NB/CNPs and other pathogen infections. This data indicate that levels of Nanobacteria IgG sero-positivity are associated with the high scores of coronary calcification, suggesting that pathogen-related mechanisms play a role in early atherosclerosis.

### EXAMPLE 3

### Immune Response in Humans Against Biogenic and Artificially Made CaBP-HA Complexes

CaBP-HA complex is immunogenic in rabbits, as patented by Kajander et al., U.S. Pat. No. 5,135,851 incorporated herein by reference, as well as in rats and mice. As shown in Example 1 and 2, antibodies are also present in humans against CaBP-HA complex. In this Example we provide evidence that this complex is infectious and causes antibody response after being introduced into the human body. FIG. 3 describes the follow-up of a research scientist co-authoring this patent application. She was followed for 60 months and had negative antibody values using ELISA method. Thereafter, her left eye was exposed to a CNP pellet of bovine origin. The conjunctival infection route is generally considered to be an effective way of introducing pathogens into human body because there is no specific defense systems involved and there is direct access to venous system in circulation without passing the lymphatics. After the incident of exposure she became positive for antibody against CaBP-HA complex at a very high level and has remained so for many many years, see FIG. 3. Anti-Human CaBP-HA complex antibodies were measured as described above and anti-Human Prothrombin F1-HA antibodies were measured as described above. The dilution factor refers to sample dilution. The high test results with high dilution indicate a strong antigen-antibody response against specific antigen, in this case, human protein-HA complex, see FIG. 4, whether biogenic human origin or made synthetically.

### EXAMPLE 4

### A New Anti-inflammatory Anti-infectious Treatment

An experimental therapy was performed by Dr. Kajander (Kajander et al.) using a novel combination of the FDA approved drugs Tetracycline HCL and Etidronate (a bisphosphonate, tradename Didronate®), both having a long history of safety and efficacy at prescribed dosage levels for the treatment of, respectively, bacterial infections and osteoporosis/Pagets Disease.

Thirteen patients were identified with chronic diseases involving inflammation and potential infectious agents, for instance, Atherosclerosis and its complications, kidney stones, gall stones, other stones; pathological calcification, calciphylaxis, osteoporosis, Paget's disease, and autoimmune diseases: rheumatoid arthritis, scleroderma psoriasis and vitiligo. All patients were adults, not pregnant or planning pregnancy, and not suffering from HIV or other forms of immunodeficiency. Patients all had normal kidney function.

The drug regimen comprised of 13 weeks of therapy. Week one: Didronate® 400 mg by mouth once a day, Tetracycline 500 mg by mouth once a day, Pyridoxin 40-200 mg by mouth once a day. Weeks two through 13: Didronate 200 mg by mouth once a day, Tetracycline 250 mg by mouth twice a day or 500 mg by mouth once a day, Pyridoxin 40-200 mg by mouth once a day. Pyridoxin was used to support homocysteine metabolism.

Therapy follow-up: blood and urine samples were collected form patients every 2 weeks. After completion of the therapy 2 more test samples were collected at monthly intervals. A patient diary was requested to be kept with an emphasis placed on the documentation of changes in symptoms and performance.

Thirteen patients were enrolled in the trial. Twelve people saw their antibodies decreased. As a result, a mean 4.15-fold decrease in anti- CaBP-HA complex antibodies was detected, which is analogous to 80% reduction, indicating that anti-CaBP-HA complex antibodies can be used to follow the efficacy on therapy and eradication of CaBP-HA complexes. Therefore, it is indicated that anti-CaBP-HA complex antibodies can be used to monitor response to therapeutic agents and can be a useful indicator of the successful therapy for NB/CNPs and the numerous diseases caused thereby.

### EXAMPLE 5

### Disease Association with CaBP-HA Complex and Antibodies Thereof

The present example examined whether antibodies against NB/CNPs are associated with various diseases involving pathological calcification. 600 serum samples were acquired commercially from Clinomics Bioscience, Watervliet, N.Y. Serum antibodies were detectable in all disease groups except for the serum samples collected from patients with Crohn's Disease. Mean anti-CNP/NB antibody values were statistically greater for all disease groups versus Crohn's Disease. These data indicate that levels of anti-CNP/NB antibodies are associated with numerous diseases including Alzheimers, Aortic Stenosis, Carotid Artery Stenosis, Coronary Artery Disease, Chronic Prostatitis, Prostate Cancer, Breast Cancer, Ovarian Cancer, Kidney Stone Disease, Parkinson's Disease, Polycystic Kidney Stone Disease, Rheumatoid Arthritis, Pancreatitis, and Cholecystitis and may be an early warning sign of disease. See FIG. 5.

What is claimed is:

1. A composition comprising at least one hydroxy apatite (HA) and at least one bound calcium binding protein and/or fragment thereof (CaBP), wherein said at least one hydroxy apatite (HA) and at least one bound calcium binding protein and/or fragment thereof comprise a CaBP-HA complex.

2. The composition of claim 1, wherein said at least one bound calcium binding protein and/or fragment thereof experience at least one conformational change.

3. The composition of claim 2, wherein said conformational change is at least one of a primary conformational change and a secondary conformational change.

4. The composition of claim 2, wherein said conformational change is a primary conformational change.

5. The composition of claim 2, wherein said conformational change is a secondary conformational change.

6. The composition of claim 2, wherein said conformational change results in an immunological response from a host mammal.

7. The composition of claim 6, wherein said immunological response comprises the creation of antibodies in response to said composition.

8. The composition of claim 1, wherein said wherein said at least one hydroxy apatite (HA) and at least one bound calcium binding protein and/or fragment thereof is at least one of: proteins with a GLA-containing domain, clotting factor II, clotting factor VII, clotting factor IX, clotting factor X/Xa, tissue factor-clotting factor VIIa complex, prothrombinase complex (factor V, Xa, II), fragments of factor II, thrombin, prothrombin Fragment 1, matrix GLA-protein and osteocalcin, osteopontin, osteonectin, and proteins factor XIIIa, Fetuin A, calmodulin, Tissue Transglutaminase II, MMP-9, MMP-3, CD 42b, NF-kappa B, CD14, Fetuin B, CD40, myeloperoxidase, Fibronectin, tissue factor, human complement 5b-9, CRP , CD61, Kappa Light Chain, Macrophage L1 Protein, hsp 60, fibrillin-1, Beeta-2-microglobulin, CD 18, laminin, antitrypsin, Notch-1, BSA, LPS-binding protein (LBP), PTX3, complement C5, fibrin/fibrinogen, D-Dimer, factor V, antichymotrypsin, Annexin V, vitronectin, thrombin, Troponin T, vimentin, tropomyosin, Human Serum Albumin, Troponin I cardiac, Apo A1, MHC class I, Amyloid P protein, sCD40 L, Kallikreins, ATIII, Factor VIII, Heparan Sulphate, Factor XI, c-jun, Fra-2, Fra-1, Jun B, P-c-Jun, Transglutaminase3, alpha fetoprotein, Prostate Specific Antigen (PSA), erbB2, VEGF, alpha synuclein, and other molecules interacting and binding to such complexes, such as LPS and its component Lipid A, Thomsen-Friedenreich antigen, and modifications of proteins such as isopeptide bond made by transglutaminase.

9. The composition of claim 2, wherein said conformational changes are mediated by said hydroxy apatite.

10. The composition of claim 1, wherein said at least one bound calcium binding protein and/or fragment thereof is mediated by a free, di-cationic calcium in a biological sample of a mammal.

11. The composition of claim 2, wherein said at least one conformational change is initiated by transglutaminase.

12. The composition of claim 1, wherein said at least one conformational change results in the formation of neoepitopes.

13. The composition of claim 1, wherein said composition behaves as an infective agent in a mammalian host.

14. The composition of claim 1, wherein said composition behaves as an infective agent in a mammalian host and wherein the mammal creates antibodies to at least one conformationally changed calcium binding protein and/or fragments thereof and wherein said antibodies are able to be measured using standard immunologic testing methods.

15. A method for detecting antibodies to calcium binding proteins that have undergone at least one conformational change in response to being bound to calcium phosphate hydroxy apatite particles in a biological sample from a mammal comprising (i) providing a CaBP-HA complex, (ii) contacting a biological sample from said mammal with said CaBP-HA complex for a time and under conditions for an antibody-antigen complex to form, then (iii) detecting whether a complex forms between said antibody and said CaBP-HA complex..

16. The method of claim 15, wherein said CaBP-HA complexantigen, or an antibody-binding fragment thereof, is from a human source.

17. The method of claim 15, wherein said CaBP-HA complex is from a bovine source.

18. The method of claim 15, wherein said CaBP-HA complex is from a mammalian source.

19. The method of claim 15, wherein detecting whether a complex forms between said antibody and said CaBP-HA complex is determined by immunoassay.

20. The method of claim 15, wherein detecting whether a complex forms between said antibody and said CaBP-HA complex is determined by ELISA.

21. The method of claim 15, wherein detecting whether a complex forms between said antibody and said CaBP-HA complex is determined by chemiluminescent assay.

22. The method of claim 15, wherein detecting whether a complex forms between said antibody and said CaBP-HA complex is determined by immunoturbidometry.

23. The method of claim 15, wherein detecting whether a complex forms between said antibody and said CaBP-HA complex is determined by immuno bead methods.

24. The method of claim 15, wherein detecting whether a complex forms between said antibody and said CaBP-HA complex is determined by a lateral flow fast assay based on antibody-antigen complex forming a visible band.

25. The method of claim 15, wherein said biological sample is at least one of serum or plasma, whole blood, cell culture samples, cerebrospinal fluid, urine, saliva, semen, amniotic fluid and cyst fluid.

26. The method of claim 15, wherein said biological sample is urine.

27. The method of claim 15, wherein said biological sample is serum.

28. The method of claim 15, wherein said biological fluid is at least one of mucous and saliva.

29. The method of claim 15, wherein said biological sample is taken from a human patient.

30. The method of claim 15, wherein said CaBP-HA complex is immobilized by being bound to a solid surface.

31. The method of claim 15, wherein said CaBP-HA complex is prepared using commercially available hydroxy apatite or hydroxy apatite synthesized according to standard protocols that is thereafter subjected to the serum of a mammal, allowed to incubate from approximately 1 minute to approximately 1 month in said serum or purified protein or a mixture of purified proteins, wherein the hydroxy apatite binds calcium binding proteins contained in the serum, or added purified proteins, allowing for the formation of a collection of primary and secondary conformationally changed calcium binding proteins bound to said hydroxy apatite.

32. The method of claim 15, wherein said CaBP-HA complex is derived from NB/CNPs collected from a mammal comprised of said CaBP-HA biomineral NB/CNP-CaBP-HA complex.

33. The method of claim 31, wherein at least one cross-linking enzymes is added.

34. The method of claim 33, wherein said at least one cross-linking enzyme is transglutaminase.

35. The method of claim 33, wherein the addition of said at least one cross-linking enzyme results in the formation of neoepitopes.

36. The method of claim 32, wherein at least one cross-linking enzymes is added.

37. The method of claim 36, wherein said at least one cross-linking enzyme is transglutaminase.

38. The method of claim 36, wherein the addition of said at least one cross-linking enzyme results in the formation of neoepitopes.

39. The method of claim 31, wherein said purified proteinor protein mixture is at least one of: proteins with a GLA-containing domain, clotting factor II, clotting factor VII, clotting factor IX, clotting factor X/Xa, tissue factor-clotting factor VIIa complex, prothrombinase complex (factor V, Xa, II), fragments of factor II, thrombin, prothrombin Fragment 1, matrix GLA-protein and osteocalcin, osteopontin, osteonectin, and proteins factor XIIIa, Fetuin A, calmodulin, Tissue Transglutaminase II, MMP-9, MMP-3, CD 42b, NF-kappa B, CD14, Fetuin B, CD40, myeloperoxidase, Fibronectin, tissue factor, human complement 5b-9, CRP , CD61, Kappa Light Chain, Macrophage L1 Protein, hsp 60, fibrillin-1, Beeta-2-microglobulin, CD 18, laminin, antitrypsin, Notch-1, BSA, LPS-binding protein (LBP), PTX3, complement C5, fibrin/fibrinogen, D-Dimer, factor V, antichymotrypsin, Annexin V, vitronectin, thrombin, Troponin T, vimentin, tropomyosin, Human Serum Albumin, Troponin I cardiac, Apo A1, MHC class I, Amyloid P protein, sCD40 L, Kallikreins, ATIII, Factor VIII, Heparan Sulphate, Factor XI, c-jun, Fra-2, Fra-1, Jun B, P-c-Jun, Transglutaminase3, alpha fetoprotein, Prostate Specific Antigen (PSA), erbB2, VEGF, alpha synuclein, and other molecules interacting and binding to such complexes, such as LPS and its component Lipid A, Thomsen-Friedenreich antigen, and modifications of proteins such as isopeptide bond made by transglutaminase.

40. The method of claim 31, wherein said purified protein or protein mixture is a mammalian prothrombin or at least one fragment thereof.

41. The method of claim 31, wherein said purified protein or protein mixture is from a human source

42. The method of claim 31, wherein said purified protein or protein mixture is from a bovine source.

43. The method of claim 15, wherein detecting whether a complex forms between said antibody and said CaBP-HA complex comprises incubating said complex with a second antibody specific for the bound antibody, said second antibody being linked to a reporter molecule which is used to indicate the binding of the second antibody to the antibody:immobilized antigen complex.

44. The method of claim 43, wherein said second antibody may comprise anti-human or anti-specific species of animal immunoglobulins, or their specific subgroups IgG, IgM, IgA, or IgE, or a mixture thereof.

45. The method of claim 43, wherein the reporter molecule provides an identifiable signal which allows the detection of binding of the second antibody to the antibody-immobilized antigen complex.

46. The method of claim 15, wherein the detecting whether a complex forms between said antibody and said CaBP-HA complex is useful in the diagnosis, prognosis and assessment of at least one disease associated with pathological calcification.

47. The method of claim 46, wherein said disease is at least one of Arteriosclerosis, Atherosclerosis, Coronary Heart Disease, Coronary Artery Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases, Lupus, Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease) and Parkinson's Disease.

48. A composition or comprising at least one hydroxy apatite (HA), lipopolysaccharide binding protein (LPSBP) and at least one calcium binding protein (CaBP), wherein said at least one hydroxy apatite (HA), lipopolysaccharide binding proteins (LPSBP) and at least one calcium binding proteins (CaBP) comprise a HA-LPSBP-CaBP complex.

49. The composition of claim 48, wherein said at least one lipopolysaccharide binding protein and said at least one calcium binding protein experience at least one conformational change.

50. The composition of claim 49, wherein said conformational change is at least one of a primary conformational change and a secondary conformational change.

51. The composition of claim 49, wherein said conformational change is a primary conformational change.

52. The composition of claim 49, wherein said conformational change is a secondary conformational change.

53. The composition of claim 48, wherein said composition behaves as an endotoxin in a mammalian host.

54. The composition of claim 8, wherein antibodies to said composition can be detected by immunoassay.

55. A process for using a composition comprising at least one hydroxy apatite and at least one bound calcium binding protein for the detection of anti-CaBP-HA antibodies in a mammal comprising the steps of:
a. preparing at least one hydroxy apatite;
b. subjecting said at least one hydroxy apatite to the serum of a mammal;
c. allowing said at least one hydroxy apatite and serum to incubate approximately 1 minute to approximately 1 month in serum or purified protein or a mixture of purified proteins;
d. allowing said at least one hydroxy apatite to bind calcium binding proteins contained in said serum or purified protein or a mixture of purified proteins;
e. allowing for the formation of a collection of primary and secondary conformationally changed calcium binding proteins bound to said hydroxy apatite.

56. An enzyme linked immunosorbent assay (ELISA) kit to detect anti-CaBP-HA antibodies in a biological sample, the kit comprising:
(a) a solid support coated with clear CaBP-HA antigens;
(b) a biological sample from a mammal;
(c) a second antibody linked to a reporter molecule reactive with anti-CaBP-HA antibodies;
(d) a calorimetric agent; and
(e) a standard.

57. A method for identification of anti-CaBP-HA antibodies in a sample comprising:
(a) providing a kit for identification for anti-CaBP-HA antibodies comprising isolated antigens and secondary antibodies, reagents and apparatus for detection of antibody binding reaction in a test sample and mixing the isolated antibodies with the sample, and
(b) detecting whether there is an antibody binding reaction in the sample so as to ascertain the presence of anti-CaBP-HA antibodies.

58. The method according to claim 28, further comprising adding a calcium chelator to said reagents.

59. The method according to claim 28, wherein said CaBP-HA complex is associated with one or more of Arteriosclerosis, Atherosclerosis, Coronary Heart Disease, Coronary Artery Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases, Lupus, Erythematosus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease) and Parkinson's Disease.

60. The method according to claim 28 wherein said presence of anti-CaBP-HA complex antibodies is associated with calcification-related diseases.

61. The method according to claim 28 wherein said presence of anti-CaBP-HA complex antibodies is associated with levels of coronary calcification.

62. The method according to claim 28 wherein said presence of anti-CaBP-HA complex antibodies identifies patients at highest risk for future cardiac event.

63. The method according to claim 28 wherein said presence of anti-CaBP-HA complex antibodies identifies patients at risk for coronary artery calcification.

64. The method according to claim 28 wherein said presence of anti-CaBP-HA antibodies identifies patients at risk for Coronary Artery Disease.

65. The method according to claim 28 wherein said presence of anti-CaBP-HA complex antibodies identifies patients at risk for one or more of Arteriosclerosis, Atherosclerosis, Coronary Heart Disease, Coronary Artery Disease, Chronic Heart Failure, Valve Calcifications, Arterial Aneurysms, Calcific Aortic Stenosis, Transient Cerebral Ischemia, Stroke, Peripheral Vascular Disease, Vascular Thrombosis, Dental Plaque, Gum Disease (dental pulp stones), Salivary Gland Stones, Chronic Infection Syndromes such as Chronic Fatigue Syndrome, Kidney and Bladder Stones, Gall Stones, Pancreas and Bowel Diseases (such as Pancreatic Duct Stones, Crohn's Disease, Colitis Ulcerosa), Liver Diseases (such as Liver Cirrhosis, Liver Cysts), Testicular Microliths, Chronic Calculous Prostatitis, Prostate Calcification, Calcification in Hemodialysis Patients, Malacoplakia, Autoimmune Diseases, Erythematosus, Lupus, Scleroderma, Dermatomyositis, Antiphospholipid Syndrome, Arteritis Nodosa, Thrombocytopenia, Hemolytic Anemia, Myelitis, Livedo Reticularis, Chorea, Migraine, Juvenile Dermatomyositis, Grave's Disease, Hypothyreoidism, Type 1 Diabetes Mellitus, Addison's Disease, Hypopituitarism, Placental and Fetal Disorders, Polycystic Kidney Disease, Glomerulopathies, Eye Diseases (such as Corneal Calcifications, Cataracts, Macular Degeneration and Retinal Vasculature-derived Processes and other Retinal Degenerations, Retinal Nerve Degeneration, Retinitis, and Iritis), Ear Diseases (such as Otosclerosis, Degeneration of Otoliths and Symptoms from the Vestibular Organ and Inner Ear (Vertigo and Tinnitus)), Thyroglossal Cysts, Thyroid Cysts, Ovarian Cysts, Cancer (such as Meningiomas, Breast Cancer, Prostate Cancer, Thyroid Cancer, Serous Ovarian Adenocarcinoma), Skin Diseases (such as Calcinosis Cutis, Calciphylaxis, Psoriasis, Eczema, Lichen Ruber Planus), Rheumatoid Arthritis, Calcific Tenditis, Osteoarthritis, Fibromyalgia, Bone Spurs, Diffuse Interstitial Skeletal Hyperostosis, Intracranial Calcifications (such as Degenerative Disease Processes and Dementia), Erythrocyte-Related Diseases involving Anemia, Intraerythrocytic Nanobacterial Infection and Splenic Calcifications, Chronic Obstructive Pulmonary Disease, Broncholiths, Bronchial Stones, Neuropathy, Calcification and Encrustations of Implants, Mixed Calcified Biofilms, and Myelodegenerative Disorders (such as Multiple Sclerosis, Lou Gehrig's and Alzheimer's Disease) and Parkinson's Disease.

66. A particle that behaves as an infective agent in a mammalian host by the formation of endotoxin-containing particle comprising hydroxy apatite, lipopolysaccharide-binding proteins, and CaBPs (“HA-LPSBP-CaBP”).

67. The particle of claim 35, wherein detection of anti-HA-LPSBP-CaBP antibodies of said particle via immunoassay.

68. A method for creating CaBP-HA complexes by incubating hydroxy apatite with human or bovine serum or purified prothrombin or its fragments for the detection of anti-serum or anti-prothrombin antibodies.

69. Detection of covalently modified neoepitopes in sera or purified proteins in association with hydroxy apatite.

70. Screening of blood and tissue donors for presence of antibodies against CaBP-HA complexes which could be potentially harmful for the recipient.

71. Detection of anti-CaBP-HA antibodies for a pooling donors and purifying antibodies for diagnostic and therapeutic purposes.

72. Enrichment of B- and T-lymphocytes recognizing CaBP-HA complexes by exposing blood samples to said complex surfaces and isolating cells bound to surfaces due to their antibodies or T-cell receptors specifically recognizing the complexes.

73. Elimination of said cells in previous claim 43 by binding them to the complexes immobilized on suitable surfaces.

74. Immunizing or immunomodulating humans or animals with CaBP-HA complexes to elicit blocking and therapeutic immunological responses.

75. Passive protecting of humans or animals exposed to infectious CaBP-HA complexes using anti-CaBP-HA complex antibodies.

76. A vaccination against infectious CNPs using non-infectious forms of CaBP-HA complexes.
